(19) United States
(12) Patent Application Publication
(10) Pub. No.: US 2016/0280624 A1
(43) Pub. Date: Sep. 29, 2016

(54) TERTIARY ALKYLAMINES AS METHACROLEIN SYNTHESIS CO-CATALYST

(71) Applicants: Rudolf BURGHARDT, Darmstadt (DE); Steffen KRILL, Muehltal (DE); Torsten BALDUF, Pfungstadt (DE); Eduard RUNDAL, Frankfurt (DE)

(72) Inventors: Rudolf BURGHARDT, Darmstadt (DE); Steffen KRILL, Muehltal (DE); Torsten BALDUF, Pfungstadt (DE); Eduard RUNDAL, Frankfurt (DE)

(73) Assignee: EVONIK ROEHM GMBH, Darmstadt (DE)

(21) Appl. No.: 15/037,171

(22) PCT Filed: Dec. 3, 2014
(86) PCT No.: PCT/EP2014/076326

§ 371 Date: May 17, 2016

(30) Foreign Application Priority Data
Dec. 12, 2013 (EP) 13196810.9

(51) Int. Cl. C07C 45/75; C07C 67/39

(52) U.S. Cl. CPC C07C 45/75; C07C 67/39

(57) ABSTRACT

The present invention relates to a process for producing methacrolein from propionaldehyde and formalin. This novel process is notable in that the catalyst system used for this reaction is modified by the simultaneous presence of secondary and tertiary amines such that the synthesis can be carried out more efficiently and with less purification being required.

The present invention relates to an improved process for producing methacrolein from propionaldehyde and formalin. This novel process is notable in that the catalyst system used for this reaction is modified such that the synthesis can be carried out more efficiently and with less purification being required.

Methacrolein is used in chemical synthesis particularly as an intermediate for producing methacrylic acid, methyl methacrylate, or else active ingredients, odourants or flavourings. There is a great deal of interest in very simple, economical, and environmentally friendly production processes.

### PRIOR ART

The present invention relates to the modification of the industrially important process for producing methacrolein from propanal and formaldehyde or formalin. The reaction is effected by means of a Mannich reaction. Such a process for producing methacrolein is described, inter alia, in the printed publications DE 32 13 681, U.S. Pat. No. 7,141,702, U.S. Pat. No. 4,408,079, JP 3069420, JP 4173757, EP 0 317 909 and U.S. Pat. No. 2,848,499.

In this process, propionaldehyde is reacted with formalin in the presence of dimethylamine and acetic acid as homogeneous catalysts at temperatures of about 160° C. to 180° C. and a with residence time of about 10 sec. The reaction mixture is distillatively separated. A methacrolein-water heteroazeotrope is obtained at the top of the column. An aqueous solution comprising the homogeneous catalysts, traces of methacrolein and high boilers is removed at the bottom of the column. A portion of the aqueous effluent, for example about 50%, can optionally be recycled.

Under the Eschweiler-Clarke reaction conditions, in a Leuckart-Wallach-like reaction, the catalytically active amines required for the reaction form more highly alkylated derivatives, which are no longer catalytically active according to the prior art. Thus, for example, dimethylamine reacts with one equivalent of formaldehyde to form trimethylamine, while releasing water. This trimethylamine (TMA) formed from dimethylamine (DMA) during the reaction is catalytically inactive according to DE 32 13 681. U.S. Pat. No. 4,408,079 additionally states that triethylamine reduces the selectivity of the reaction and must therefore be removed. However, such removal results in higher overall catalyst usage in a continuous operation. Thus, the TMA cannot be removed without simultaneous removal of DMA and both the excessively removed fractions of DMA and the DMA reacted to give TMA need to be replaced. Furthermore, this is a distillative removal and the low-boiling methacrolein removed as well therefore needs to be recovered in a costly and inconvenient further distillation step.

According to the prior art, it is consequently necessary to increase the DMA charge when recycling the bottom stream in order to compensate for the loss of catalytic activity due to TMA formation.

### PROBLEM

The problem addressed by the present invention in view of the prior art was accordingly that of reducing, compared to the prior art, the amount of amines used while carrying out a continuously operated amine-catal reaction for methacrolein syntheis.

The problem addressed was, in particular, that of providing a process in which fewer purification steps are required and the amount of waste water can be reduced.

Further problems addressed but not explicitly referred to will become apparent from the overall context of the description which follows and the claims.

### SOLUTION

The process according to the invention is used to optimize the amine-containing components of the catalyst system of a—preferably continuously operated—Mannich reaction wherein propanal is reacted with formaldehyde to give methacrolein.

These problems are solved by a novel process in which propanal is continuously reacted with formaldehyde at a temperature of from 100° C. to 300° C. and at a pressure of from 5 bar to 100 bar in the presence of from 0.1 to 30 mol %, preferably from 0.5 to 20 mol %, of organic base and from 0.1 to 30 mol %, preferably from 0.5 to 20 mol %, of acid, in each case based on propanal, to give methacrolein. The formaldehyde component is to be understood as being independent of the charging form and the term also comprises formalin and paraformaldehyde in relation to the charge.

The process according to the invention is, in particular, notable in that the organic base is a mixture of a secondary alkylamine and a tertiary alkylamine in a molar ratio of between 20 to 1 and 1 to 3, preferably between 10 to 1 and 1 to 2, more preferably between 7 to 1 and 1 to 1, and most preferably between 5 to 1 and 2.5 to 1.

In a first embodiment, the secondary and tertiary alkylamines are charged to the plant, preferably directly to the reactor, as a mixture. Here, the ratio of the two components is either as indicated or there is a smaller proportion of tertiary alkylamines at the inlet of the reaction compared to the reactor outlet since these form spontaneously during the reaction.

In a preferred alternative embodiment of the invention, only secondary alkylamines are charged to the plant. Here, the tertiary alkylamines form during operation of the plant and accumulate on recirculation. In this embodiment, it is particularly preferable for the tertiary alkylamine content to be determined continuously or regularly, for example by sampling. Based on this determination, the amine-containing catalyst solution is replaced as appropriate when the ratio of secondary to tertiary amine components fails below 1 to 3, preferably belowl to 2 and more preferably belowl to 1. This replacement is effected by discharging the aqueous reaction phase and/or by charging fresh secondary alkylamine to the system, preferably directly into the reactor. To this end, the plant is designed such that the reaction mixture is continuously removed from the reaction space and separated via a phase separator or preferably via a distillation column. The organic phase comprising the methacrolein is obtained, for example as distillate, and discharged. All or some of the residual aqueous distillation bottom product, containing inter alia methacrolein residues, is returned to the reaction space. At this point, portions of the aqueous phase can be withdrawn to replace the catalyst components, in particular the amine components. Withdrawal via a phase separator is effected similarly. Withdrawal not only replaces the catalyst components but also simultaneously reduces the amount of water and discharges by-products that have been formed from the system.

The reaction solution to be worked up can be supplied from the reaction space into the distillation column above, within or below the separation section of the column. A bottom product collects in the vaporizer section of this column, said bottom product consisting predominantly of water of reaction and, as the case may be, comprising the amounts of water entering the recirculating process with the formalin solution. This bottom product further comprises the catalyst components, for example the organic acid and the secondary amine and the tertiary amine and/or the salts formed therefrom, and by-products of the reaction, or the combination of the two. This aqueous catalyst solution can preferably be drawn off below the feed point to the column at the bottom of the column. The total water present is made up of the water charged as catalyst solution, the water formed during the reaction and optionally the water from the formaldehyde solution. Further, though less significant, sources of water are constituents of the technical-grade reactants such as propanal, and water formed in various side reactions of the catalyst components with reactants, by-products and reaction products, and also water of reaction from all these components formed under the reaction conditions.

It is also possible to divide the bottom output into two substreams such that one substream carries the exact amount of water formed in the reaction and introduced with the starting materials. This substream is then discharged and the remaining fraction is returned to the reactor. The discharged portion would, for example, be disposed of thermally or worked up biologically in this procedure.

The aqueous phase of the reaction withdrawn continuously, semi-continuously or batchwise in this way is then preferably discharged, optionally separated into two phases via a membrane and the amine-containing waste water thus obtained (retentate) is incinerated in a thermal oxidizer, Prior to incineration, the waste water can be still further separated via an additional distillation in order thereby to convey co-discharged product, for example, back into the reaction circuit or the product work-up, Some or all of the retentate of the membrane step can be passed back into the reactor or the work-up.

It is particularly preferable to discharge the aqueous phase of the reaction and separate it into two phases via a membrane. The amine-containing waste water thus obtained is then incinerated in a thermal oxidizer or otherwise disposed of, for example in a biological work-up, and some of the retentate of the membrane separation is returned to the reactor. It is also possible to further concentrate the retentate in a distillation step. Some of the concentrate thus obtained can then be passed back into the reactor.

As an alternative to membrane separation, the aqueous phase of the reaction can be discharged and separated in a distillation. The distillation bottom product thus obtained is then passed back into the reactor. The aqueous phase is discharged for disposal. This disposal may be incineration in a thermal oxidizer or a biological work-up.

On the other hand, it is particularly preferable for the methacrolein to be converted, after a purification step, into methacrylic acid or methyl methacrylate in a second process which continuously follows the process.

The Mannich-reaction-based processes suitable for producing methacrolein are known to the person skilled in the art and are the subject of corresponding review articles, for example in Ullmann's Encyclopedia of Industrial Chemistry 2012, Wiley-VCH Verlag GmbH & Co. KGaA, Weinheim, Acrolein and Methacrolein. DOI: 10.1002/14356007.a01_149.pub2. In particular, the process carried out with particular preference in accordance with the invention prior to the feeding-in of the amine-containing water of reaction relates to a continuously conducted Mannich reaction as disclosed in the European patent application having the application reference number 13002076.1. According to the invention, such a preferred preliminary step is illustrated by reference to the disclosure-content of this application relating to the methacrolein synthesis.

Such a particularly preferred Mannich reaction is carried out in the presence of from 0.1 to 20 mol % of organic base, preferably a secondary amine, and 0.1 to 20 mol % of acid, in each case based on the propanal used, at a temperature of from 100° C. to 300° C. and at a pressure of from 5 to 100 bar.

The acids are generally inorganic acids or organic monocarboxylic, dicarboxylic or polycarboxylic acids, preferably monocarboxylic acids, in particular aliphatic monocarboxylic acids. Particular preference is given to using at least one organic acid, more preferably acetic acid, for reaction with propanal and formaldehyde. The proportion of acid is between 0.1 and 20 mol %, advantageously from 0.5 to 10 mol %, preferably 1 to 5 mol %, based on propanal.

Useful inorganic acids include, in particular, sulphuric acid, phosphoric acid, hydrochloric acid, carbonic acid and boric acid. In addition to acetic acid, useful organic acids also include formic acid, propionic acid, isobutyric acid, n-butyric acid, oxalic acid, adipic acid, caprylic acid, phenylformic acid and 2-ethylhexanoic acid. In particular, mixtures of two or more acids can also be useful. Mixtures of organic and inorganic acids, in particular of acetic acid and sulphuric acid, are also of good suitability.

It is preferable to use an organic acid or a mixture of one organic acid and sulphuric acid. It is particularly preferable to use only formic acid, acetic acid or propionic acid.

Useful secondary amines supplied to the system as catalyst include for example: dimethylamine, diethylamine, methylethylamine, methylpropylamine, dipropylamine, dibutylamine, diisopropylamine, diisobutylamine, methylisopropylamine, methylisobutylamine, methyl-sec-butylamine, methyl(2-methylpentyl)amine, methyl(2-ethylhexyl)amine, pyrrolidine, piperidine, morpholine, N-methylpiperazine, N-hydroxyethylpiperazine, piperazine, hexamethyleneimine, diethanolamine, methylethanolamine, methylcyclohexylamine, methylcyclopentylamine, dicyclohexylamine or corresponding mixtures. The secondary amine is preferably dimethylamine or diethylamine, more preferably dimethylamine.

Useful tertiary amines supplied to the system or formed during the process include for example: trimethylamine, triethylamine, dimethylethylamine, diethylmethylamine, dimethylpropylamine, dipropylmethylamine, tripropylamine, tributylamine, triisopropylamine, triisobutylamine, dimethylisopropylamine, diisopropylmethylamine, dimethylisobutylamine, diisobutylmethylamine, triethanolamine, dimethylethanolamine, diethanolmethylamine, dimethylcyclohexylamine, dicyclohexylmethylamine, dimethylcyclopentylamine, dicyclopentylmethylamine, tricyclohexylamine or corresponding mixtures. The tertiary amine is preferably trimethylamine or triethylamine, more preferably trimethylamine.

It is very particularly preferable when the secondary alkylamine is dimethylamine and, simultaneously, the tertiary alkylamine is trimethylamine.

The proportion of organic base is between 0.1 and 30 mol %, advantageously from 0.5 to 20 mol %, preferably 1 to 10 mol %, based on propanal.

The ratio of the equivalents of amine to acid is preferably selected such that the resulting pH of the reaction mixture prior to the reaction is from 2.5 to 9.

In particular, it is a great advantage of the present invention that the MAL synthesis can be carried out with an amount of waste water which is distinctly reduced compared to the prior art. Since the tertiary alkylamine surprisingly need not be continuously removed from the system and, simultaneously, the secondary needs replacement to a lesser extent, the continuous production process can be carried out with this reduced amount of waste water.

This has the further advantage that the number of purification steps is simultaneously reduced. This applies in particular to the waste water which is considerably more concentrated on discharge.

The process according to the invention has the great advantage that it can be carried out using relatively simple and inexpensive plants or in existing plants. The plants are simple to service and inexpensive to maintain.

The examples which follow serve to more particularly describe preferred embodiments of the present invention without any intention that this should limit the invention.

### EXAMPLES

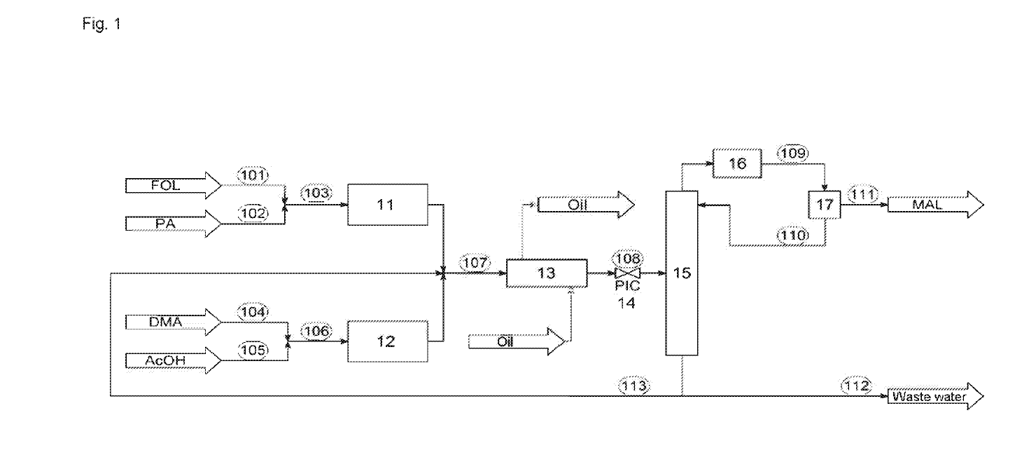

In a plant according to FIG. 1, propanal (PA) is continuously reacted with formaldehyde using dimethylamine (DMA) or a DMA-TMA mixture and a 2.8 to 1 mixture of acetic acid (AcOH) and sulphuric acid. The amine-proton ratio was adjusted to 0.75. Sulphuric acid is, of course, given double weighting compared to acetic acid when calculating this ratio. The amounts of amine used are apparent from Table 1.

251 g/h of PA and 349 g/h of 37 per cent formalin are homogeneously premixed (molar ratio of 1:1). The catalyst solution consisting of the amines and acids mentioned is fed into the preheater (12). Both streams are heated to a temperature of 170° C. before being combined. The preheated streams are combined in a T-mixer directly connected to a flow tube reactor ( 1/16 inch tube with a length of 4.2 m). The reactor is thermostated with an oil bath operated at 180° C.; the residence time is less than 10 s (see Table 1); the pressure in the tubular reactor is 70 bar. Downstream of the tubular reactor, the mixture is decompressed in valve (14) and supplied to the column (15). 335 g/h of the bottom output are returned to the reactor (13), 370 g/h of the bottom output disposed of as waste water. After condensation of the top stream in condenser (16) and phase separation in (17), a methacrolein-rich phase having a methacrolein content of 96.5% is discharged as product (111) and the aqueous output of the phase separator is returned to the column (15). The propionaldehyde conversion and the yield based on the propionaldehyde used are shown in Table 1.

The reactions of Examples 3 to 5 and Comparative Examples (CE) 4 and 5 differ in that they were carried out at an oil bath temperature of 160° C. instead of 180° C. In addition, the acid used in these examples/comparative examples was exclusively acetic acid and not a mixture of sulphuric acid and acetic acid. The amine-proton ratio was adjusted to 0.91.

TABLE 1

| | Amount of | Amount of | | Con- | |
|---|---|---|---|---|---|
| | DMA mol/ | TMA mol/ | Residence | version | Yield |
| | mol of PA | mol of PA | time/s | of PA/% | of MAL/% |
| Ex. 1 | 0.029 | 0.011 | 8.6 | 99.8 | 95.6 |
| Ex. 2 | 0.022 | 0.018 | 8.6 | 91.4 | 96.7 |
| CE1 | 0.040 | 0 | 8.6 | 99.9 | 95.4 |
| CE2 | 0.029 | 0 | 8.6 | 98.7 | 96.7 |
| CE3 | 0.019 | 0 | 8.6 | 82.7 | 92.7 |
| Ex. 3 | 0.039 | 0.0068 | 9.5 | 99.2 | 97.0 |
| Ex. 4 | 0.039 | 0.0136 | 9.5 | 99.5 | 97.0 |
| CE4 | 0.039 | 0 | 9.5 | 98.4 | 96.5 |
| Ex. 5 | 0.061 | 0.047 | 9.5 | 99.9 | 98.1 |
| CE5 | 0.060 | 0 | 9.5 | 99.9 | 98.0 |
| Ex. 6 | 0.062 | 0.024 | 9.7 | 99.9 | 98.0 |
| Ex. 7 | 0.064 | 0.064 | 9.4 | 99.9 | 98.0 |
| Ex. 8 | 0.065 | 0.084 | 9.3 | 99.9 | 98.0 |
| Ex. 9 | 0.080 | 0.160 | 9.5 | 99.9 | 98.0 |
| Ex. 10 | 0.064 | 0.127 | 9.7 | 99.9 | 97.6 |
| CE6 | 0.042 | 0 | 9.5 | 98.3 | 96.5 |
| Ex. 11 | 0.042 | 0.007 | 9.5 | 99.6 | 97.5 |
| Ex. 12 | 0.042 | 0.015 | 9.5 | 99.8 | 97.5 |
| Ex. 13 | 0.042 | 0.022 | 9.5 | 99.8 | 97.5 |

It is clearly apparent from Examples 1 and 2 compared to Examples CE2 and CE3 that the additional presence of TMA, with identical amounts of DMA, can distinctly enhance the yield and the conversion. A comparison of Ex.1 and CE1 even reveals the result, which is very surprising in view of the prior art, that both the yield and the propionaldehyde conversion remain almost unchanged when relatively small TMA contents of less than 40 mol % and an identical molar amine concentration are used. Only relatively high TMA contents, corresponding to Example 2, cause both to decline somewhat. Yet in this case too, both the conversion and the yield remain in a range that is of economic interest.

It is evident from Examples 3 and 4 with reference, in turn, to Comparative Example 4 that accumulation of TMA in the reactor—simulated here by additional amounts in the experimental set-up—surprisingly also leads to higher conversions and yields, even though selectivity appears to be minimally reduced. This small selectivity loss is, however, more than compensated by the increase in conversion.

In turn, Examples 5 and 6 and Comparative Example 5 show that, as expected, particularly high amine concentrations can be used to achieve a particularly high conversion and high MAL yields. It is, however, surprising and very much in contradiction of the prior art that these high conversions are achieved even when there is so much trimethylamine in the reactor that the ratio of trimethylamine to dimethylamine is only just under 0.8.

It is apparent from Examples 7 to 10 that surprisingly high yields and selectivities can be achieved even with equimolar amounts of TMA and DMA or with an excess of TMA. This shows, ultimately, that the present invention has overcome a long-standing prejudice of the prior art.

It is readily apparent to the person skilled in the art that these experiments are also applicable to continuous production processes where TMA accumulates.

It is again apparent from Examples CE6 and 11 to 13 that both the selectivity and the yield surprisingly increase with increasing TMA concentration at constant DMA concentration and with other process parameters constant. This leads to the conclusion not only that, contrary to the general teaching of the prior art, TMA does not disrupt the reaction, but rather that it even contributes to the catalysis of the reaction.

### LIST OF REFERENCE NUMERALS

FIG. 1: Schematic representation of a plant suitable for carrying out the process according to the invention.

FOL formalin (aqueous formaldehyde solution)

PA propanal

DMA aqueous dimethylamine solution

AcOH acetic acid

MAL methacrolein

11 heat exchanger (preheater)

12 heat exchanger (preheater)

13 reactor (tubular reactor)

14 pressure retaining valve

MAL distillation column

16 condenser

17 phase separator

101 aqueous formalin line

102 propanal line

103 line into heat exchanger

104 dimethylamine line (40% aqueous solution)

105 acetic acid line

106 line into heat exchanger

107 line into reactor

108 product mixture line to column

109 condensate line

110 return stream into column

111 MAL to step B)

112 line to waste water

113 return of bottom output

1. A process for making methacrolein, the process comprising continuously reacting propanal with formaldehyde at a temperature of from 100° C. to 300° C. and at a pressure of from 5 bar to 100 bar, thereby forming methacrolein,
wherein said continuously reacting is performed in the presence of from 0.1 to 30 mol % of organic base and from 0.1 to 30 mol % of acid, in each case based on propanal, and
wherein the organic base is a mixture of a secondary alkylamine and a tertiary alkylamine in a molar ratio between 20 to 1 and 1 to 3.

2. A process according to claim 1, wherein
the molar ratio of the secondary alkylamine to the tertiary alkylamine is between 7 to 1 and 1 to 1.

3. A process according to claim 2, wherein
the secondary alkylamine is dimethylamine and the tertiary alkylamine is trimethylamine.

4. A process according to claim 1, wherein
the alkylamines are charged to the plant as a mixture.

5. A process according to claim 1, wherein
only secondary alkylamines are charged to the plant, the tertiary alkylamines accumulate during operation of the plant, the ratio of secondary alkylamines to tertiary alkylamines is determined continuously or regularly and the amine-containing catalyst solution is replaced when the amine component ratio of secondary alkylamine to tertiary alkylamine falls below 1 to 3.

6. A process according to claim wherein
the aqueous phase of the reaction is discharged continuously, semi-continuously or batchwise, optionally separated into two phases via a membrane, the amine-containing waste water thus obtained is incinerated in a thermal oxidizer and some or all of the retentate of the membrane separation is passed back into the reactor.

7. A process according to claim 6, wherein
the aqueous phase of the reaction is discharged, separated into two phases via a membrane, the amine-containing waste water thus obtained is incinerated in a thermal oxidizer and the retentate of the membrane separation is concentrated in a distillation step and the concentrate is passed back into the reactor.

8. A process according to claim 1, wherein
the aqueous phase of the reaction is discharged, separated in a distillation, the distillation bottom product is passed back into the reactor and the aqueous phase is discharged for disposal.

9. A process according to claim 1, wherein
the methacrolein obtained is converted into methacrylic acid or methyl methacrylate in a second process which continuously follows the process.

10. A process according to claim 1, wherein
the acid used is one or more organic acids or a mixture of one organic acid and sulphuric acid. cm 11. A process according to claim 1, wherein
the acid used is formic acid, acetic acid or propionic acid.
